(19) United States
(12) Patent Application Publication
(10) Pub. No.: US 2007/0197919 A1
(43) Pub. Date: Aug. 23, 2007

(54) Video Endoscopy Device

(76) Inventors: Ingo Krisch, Bochum (DE); Werner Brockherde, Duisburg (DE); Bedrich Hosticka, Muhlheim (DE)

Correspondence Address: GLENN PATENT GROUP, 3475 EDISON WAY, SUITE L, MENLO PARK, CA, 94025, US

(21) Appl. No.: 11/625,218

(22) Filed: Jan. 19, 2007

Related U.S. Application Data
(63) Continuation of application No. PCT/EP04/08058, filed on Jul. 19, 2004.

(51) Int. Cl. A61B 6/00

(52) U.S. Cl. 600/476

(57) ABSTRACT

A video endoscopy device includes a sensor device and a catheter for routing radiation to a distal end of the catheter and for outputting same at the distal end of the catheter, and for receiving reflected radiation at the distal end and imaging same onto the sensor device, the sensor device being arranged, within the catheter, near the distal end of the catheter, and is configured to convert the reflected radiation into an electric signal, and the catheter being configured to route the electric signal to a proximal end of the catheter. The video endoscopy device thus allows improved image quality, such as with pre-, intra-, and post-operative observations at and/or in organs and vessels in an actual, i.e. blood-filled, environment.

### CROSS-REFERENCE TO RELATED APPLICATION

This application is a continuation of copending International Application No. PCT/EP2004/008058, filed Jul. 19, 2004, which designated the United States, and was not published in English and is incorporated herein by reference in its entirety.

### BACKGROUND OF THE INVENTION

1. Field of the Invention

The present invention relates to a video endoscopy device, e.g. those for depicting the interior walls of the cardiovascular system or those suitable for being used within the cardiovascular system.

2. Description of Prior Art

Even though for a few years, calls have been made for an extremely miniaturized, high-resolution endoscopy-suitable and, in particular, “blood-penetrating” camera on the part of the medical world with high priority, significant technological development steps are required for implementation, these steps still being utopian up until recently. So far, all over the world no one has yet succeeded in developing a diagnostic device which may image, through the blood, the vascular walls of the cardiovascular system with sufficiently high resolution. Since Bozzini developed the first endoscope in 1806, optical technology has made much progress and has been specialized for various applications for inspecting manifold body orifices and organs. Classical fiber endoscopy is being replaced increasingly by modern video endoscopy, since the latter guarantees considerably improved image resolution and quality. However, when used within blood vessels, both technologies have failed due to the scattering of light at the hemoglobin molecules, similar to the fact that visibility in fog is severely restricted depending on the density and size of drops. The medium of blood actually exhibits an optically opaque behavior due to the Mie scattering at the erythrocytes and/or due to the high level of absorption of the water molecules. Blood becomes sufficiently transparent for radiation within near infrared (NIR) in the range from 1.5 to 1.8 μm, as well as in the range from 2.1 to 2.3 μm, so that the use of a miniaturized NIR camera provides insights into the vascular system which has so far been detectable only with weak outlines.

With the conventional realization for representing the cardiovascular system, essentially four different methods are currently employed.

With the ultrasonic methods, both the movement of the heart may be observed and the artery and vein systems may be represented by means of a method of the Doppler technique, which is referred to as the duplex method. However, since this method actually serves for flow metering, the image resolution cannot meet the demands made by a cardiosurgeon.

In computer tomography, the activity distribution of various body layers is detected in a two-dimensional manner using emission computer tomography (ECT) following an injection of a radiopharmaceutical agent. Indeed, the concentration of TC or I within the vasculature system allows a representation of the arteries and veins, but pronounced instances of inhomogeneity and dissymmetry lead to major artifacts (misrepresentations), such as due to lung or mamma absorption in heart examinations. Due to the artifacts, the image quality of this imaging method is not adequate for heart surgery. In addition, the above-mentioned method also fails in terms of representing moving pictures.

In magnet resonance tomography, (electrocardiogram-triggered) phase contrast angiography allows a rough representation of the vascular system, but not in real time, and is part of clinical routine.

At the moment, the technology of modified balloon catheters is still being discussed and tested, however without any prospects of a sweeping success: with this method, a balloon catheter is introduced into the cardiovascular system and is pushed, by the doctor performing treatment, through the veins and on to the location of examination before the balloon catheter is blown up there by means of Ringer's solution. The transparent envelope of the balloon presses directly against the vascular wall in the process, so that an optical system integrated into the balloon can image the structure of the wall. The disadvantages of this method are complete vascular obstruction, on the one hand, and the high pressure load on the vessels, on the other hand.

From the technical point of view, classical fiber endoscopy is optimized with regard to narrow diameters in that quartz fibers having diameters of 2.8 μm are employed as light-conducting fibers. Even though very small pixels can be realized in this manner, the method exhibits several disadvantages due to the high light losses within the visible range and due to the small numerical aperture. Even though the examination location is highly illuminated, this method will only provide images of low brightness, especially as the transmission within the infrared region deteriorates as compared to the visible region.

U.S. Pat. No. 6,178,346 describes and infrared fiber endoscopy method which is registered under the trademark of Transblood Vision in the US. Due to Mie scattering at the enthrocytes and/or due to the high level of absorption of the water molecules, blood is actually opaque. The method proposed in U.S. Pat. No. 6,178,346, however, circumvents the problems by specifically selecting the infrared wavelength. In the method, radiation generated by a laser diode is coupled into a light-conducting fiber of the endoscope by means of a beam splitter, the location of examination being illuminated as a result. The light reflected from the examination location is in turn passed on to an external camera sensor via the proximal end of the catheter via the beam splitter. An advantage of the approach suggested there is the considerable level of attenuation of the optical signal containing the image-providing information, and thus the limitation of the achievable brightness of the object.

### SUMMARY OF THE INVENTION

It is the object of the invention to provide a video endoscope device which enables improved image quality, such as for pre-, intra-, and post-operative observations at and/or within organs and vessels in an actual, i.e. blood-filled, environment.

The present invention provides a video endoscopy device including:

a sensor device; and

a catheter for outputting radiation at a distal end of the catheter, and for receiving reflected radiation at the distal end, and imaging same onto the sensor device,

the sensor device being arranged, within the catheter, in the vicinity of the distal end of the catheter, and is configured to convert the radiation reflected into an electric signal, and the catheter being configured to route the electric signal to a proximal end of the catheter.

An inventive video endoscopy device includes an (image) sensor device and a catheter for routing radiation to a distal end of the catheter and for outputting same at the distal end of the catheter, and for receiving reflected radiation at the distal end and imaging same onto the sensor device. The sensor device is arranged, within the catheter, near the distal end of the catheter, and is configured to convert the reflected radiation into an electric signal. The catheter is configured to route the electric signal to a proximal end of the catheter.

The core idea on which the invention is based is to illuminate an object to be examined by means of radiation transmitted, for example, by a light-conducting fiber, while the backscatter radiation is detected by a sensor arranged within the catheter tip so as to convert the image of the object into an electric signal which may be supplied to an external image processing device via, for example, a cable or line connection. In this manner, image transmission by means of optical-fiber cables may be dispensed with, and as a consequence, the negative impacts on the image quality due to the optical attenuation of the signal, in particular on the way back from the catheter tip to the external unit, may be avoided.

### BRIEF DESCRIPTION OF THE DRAWINGS

These and other objects and features of the present invention will become clear from the following description taken in conjunction with the accompanying drawing, in which:

FIG. 1 is a schematic block diagram of a cardiovascular video endoscope device in accordance with an embodiment of the present invention;

FIG. 2 is a schematic cross-sectional view of a distal catheter tip and/or a catheter head in accordance with an embodiment of the present invention; and

FIG. 3 is a schematic stereoscopic image of an image sensor within the distal catheter tip of FIG. 2.

### DESCRIPTION OF PREFERRED EMBODIMENTS

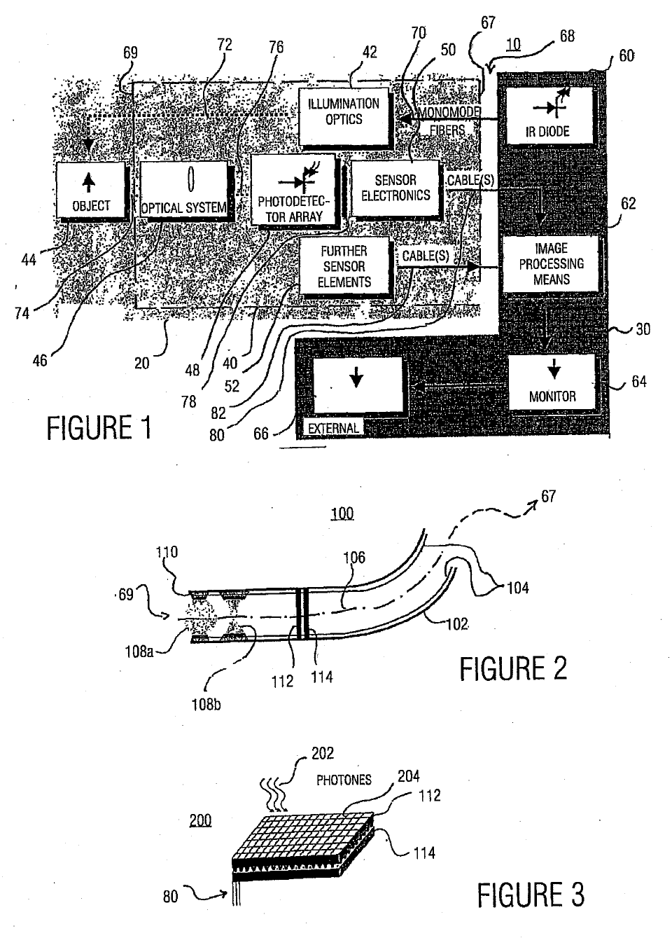

FIG. 1 shows a video endoscopy device in accordance with an embodiment of the present invention. The video endoscopy device, generally denoted by 10, is essentially subdivided into two parts, i.e. a movable part 20 and an external, static part 30.

The movable part 20 forms a movable catheter arrangement. In particular, it comprises a catheter 40 which contains optics 42 for illuminating an object 44 to be examined, such as the vascular wall of a blood vessel, an optical system 46 for imaging the illuminated object 44 onto a photodetector array, also arranged within catheter 40, as a sensor 48, a pre-processing circuit and/or sensor electronics 50 and, optionally, further sensor elements 52. Sensor electronics 50 preferably consist of a sensor drive, a readout circuit, and an image pre-processing unit.

The static part 30 essentially forms the external apparatus of video endoscopy device 10. It comprises a radiation source 60, an image and signal processing means 62, a display unit and/or a monitor 64 and a memory 66.

Catheter 40 may be coupled, at a proximal end 67 of same, to the external apparatus 30, such as via a releasable or permanent plug connection. The interface between catheter 40 and external apparatus 30 is indicted at 68 in FIG. 1. With a distal end 69 and/or a catheter tip, catheter 40 may be turned toward object 44 and/or toward the examination area to be illuminated.

A radiation router 70, such as a plurality of monomode fibers, as will be explained later on, by way of example, with reference to FIG. 2, extends within catheter 40 between proximal end 67 and optics 42 to route the radiation generated by radiation source 60 to optics 42, which homogeneously distribute said radiation onto object 44, as is indicated by a dashed line 72. Optics 42 need not be optics which are specifically provided, but may further be formed by the exit end of radiation router 70 itself. A dashed line 74 is to represent, in FIG. 1, a further radiation path between object 44 and optical system 46, specifically that radiation path on which the light reflected from the object passes into optical system 46. A third radiation path 76 is located between optical system 46 and sensor 48. On this radiation path 76, optical system 46 images object 44 onto pixel array 48, or, to put it more precisely, onto the photosensitive area of pixel array 48 which consists of an array of pixels and at a specific repetition rate generates, from the imaging, pixel measurement values for all pixels and, thus, image representations.

An electrical connection system 78 is located between sensor 48 and electronics 50 and serves to electrically connect them and/or to pass on the pixel measurement values to subsequent circuit 50. An electrical conductor 80, such as one or a plurality of cables, extends between circuit 50 and proximal end 67 of catheter 40 so as to pass on pre-processed image data obtained by circuit 50 from the pixel measurement values to image processing 62 via interface 68 in the coupled state of catheter 40. The data is thus handed over to a hardware, here image processing 62, which is external to catheter 40, via a defined interface. A further electrical conductor 82, such as one or a plurality of cables, is arranged between the optional further sensor elements 52 and image and/or signal processing 62, and/or extends therebetween, so as to pass on measurement data of sensor elements 52 to processing means 62.

As will become more obvious later on with reference to FIG. 2, optics 42, optical system 46, photodetector array 48 and circuit 50 as well as further sensor elements 52 are arranged in the vicinity of the distal end of catheter 40, and thus form the catheter head and/or a catheter tip of catheter 40.

Within the external apparatus 30, image processing 62 is connected to interface 68 for coupling proximal end 67 of catheter 40 so as to obtain, in the coupled state of catheter 40, the pre-processed data via cable 80 from pre-processing means 50, and to obtain the sensor measurement data from the optional sensors 52 via cable 82. An output of processing means 62 is connected to the input of monitor 64 so as to be able to display the image of object 44, which has been obtained within photodetector array 48, to the user of device 10 as well as to be able to display, as the case may be, current measurements results of the additional sensors 52. In addition, the output of processing means 62 is connected to memory 66 so as to be able to archive the data obtained from pre-processing means 50 and sensor elements 52, such as, for example, for subsequent evaluation of the data.

Infrared diode 60 is also connected—this time, however, in an optical manner—to interface 68 so as to be able to couple light into radiation router 70 of catheter 40 via interface 68 as soon as same is coupled to apparatus 30.

Having given the above description of the architecture of device 10, its mode of operation will be briefly described below.

In order to illuminate the examination location 44, radiation is generated externally to light source 60, which, by way of example, shall be an infrared diode below. This irradiation is then transported though catheter 40 via light conductor 70 or, in the case of the embodiment of FIG. 2, via the monomode fibers, and is homogeneously distributed onto the area to be illuminated and/or onto object 44 via optics 42. The illuminated scene 44 scatters the light back into optical system 46. This optical system 46 images the illuminated scene onto photodetector array 48 with a certain field depth range, where the image is converted into an array of pixel measurement values at a certain resolution which depends on the pixel spacing of photodetector array 48. The pixel measurement values, in turn, are passed on to sensor electronics 50 via connection system 78, sensor electronics 50 initially reading out the data and subsequently performing a certain pre-processing of the pixel measurement values which are still analog, for example, up to this point, i.e. performing, for example, pure digitalization, dynamics adjustment or the like. In order that photodetector array 48 and pre-processing means 50 can perform their tasks, they are supplied with energy via electrical connection 80. The pre-processed data is passed to image processing means 62, where the data is processed such that it is present as a video signal and may be displayed by monitor 64. Having introduced catheter 40 into the artery and vein system, a physician using device 10 may now navigate the distal end 69 and/or the image detail of optical system 46 to the desired examination location 44 while observing monitor 64.

It is possible for the physician to obtain, via further sensor elements 52, further information about the examination location 44, such as blood flow performed by a flow meter, temperature measurement performed by a temperature sensor, or the like. These measurement values may then be used for further diagnostics and control. It shall be noted that it is possible for the physician to perform, as the case may be, adjustments to pre-processing means 70 or photodetector array 48, such as an alteration of the resolution with simultaneous corresponding alteration of the image repetition rate or the like, via an input device not shown in FIG. 1, such as a keyboard.

Having described an embodiment of the present invention in rather general terms above with reference to FIG. 1, an embodiment of a catheter tip will be described in more detail below. The catheter tip shown in FIG. 2 is generally indicated at 100. With renewed brief reference to FIG. 1, catheter tip 100 of FIG. 2 is arranged at distal end 69. That part of catheter 40 which is not shown in FIG. 2 leads on to proximal end 67 of the catheter, as is indicated by a dashed part which is bent to indicate the flexibility of the catheter.

The catheter tip 100 of FIG. 2 is schematically depicted in cross section. The tubular and flexible sheath 102 of the catheter can be seen. It forms the outer jacket of the catheter. Monomode fibers 104 extend within the catheter along sheath 102 from proximal end 67 to distal end 69. In a cross section which is transverse to the longitudinal axis 106 of the catheter, they are arranged annularly around the longitudinal axis 106 along the interior wall of sheath 102. Thus, monomode fibers 104 form the radiation router 70 of FIG. 1 and transport the light of infrared diode 60 to distal end 69.

Lenses 108a and 108b are arranged at distal end 69 as a termination of the catheter in a manner such that they are axially symmetrical to longitudinal axis 106, lenses 108a and 108b forming the optical system 46 of the catheter. They are attached to the inside of sheath 102 via annular fixtures 110. It is through these fixtures 110 that monomode fibers 104 extend to be able to output their light at distal end 69. As the case may be, elements for beam expansion are provided within the fixtures 110 per monomode fiber 104. Alternatively, the terminal ends of monomode fibers 104 form optics 42 of FIG. 1 at the exit point at fixtures 110 or shortly behind. A compound arrangement of a photodetector array 112 and a semiconductor chip 114, of which the latter forms sensor electronics 50, is arranged within a specific distance behind lenses 108a-108b, i.e. in the direction of proximal end 67, transversely to the longitudinal axis 106. The compound arrangement 112, 114 is preferably also arranged within the catheter such that it is axially symmetric to the longitudinal axis 106 and attached to the interior walls of sheath 102, specifically in such a manner that the monomode fibers 104 extending on the interior wall of sheath 102 from the proximal 67 to the distal ends 69 can pass the compound arrangement 112, 114. The cables for supplying the compound arrangement 112, 114 with energy and for passing on the data from the compound arrangement 112, 114 to image processing means 62, etc., which are shown only in FIG. 1 and are omitted for clarity's sake in FIG. 2, extend within that part of the catheter which adjoins the compound arrangement 112, 114 toward the proximal end 67.

The catheter tip of FIG. 2 would be readily suited to be employed in the device of FIG. 1. Then, what would be missing in FIG. 2 in addition to the representation of the cables would only be the representation of the further sensors 52. These could be provided, for example, on the skin of sheath 102, or at the distal end 69 at the exposed side of fixture 110.

With reference to FIG. 3, an embodiment of compound arrangement 112, 114 of FIG. 2 will be described. FIG. 3 generally indicates the compound arrangement with a reference numeral 200. In FIG. 3, compound arrangement 200 is depicted in a spatial representation from a perspective wherein that side of compound arrangement 200 which is facing the distal end 69 and/or optics 108a-108b (FIG. 2), and onto which the photons which are backscattered from the object impinge onto compound arrangement 200, as is indicated by arrows 202, is visible. Compound arrangement 200 consists of photodetector array 112 and semiconductor chip 114. Photodetector array 112 is formed within a semiconductor substrate, such as within a III-V semiconductor, such as within an InGaAs semiconductor. Photodiodes are formed within the semiconductor substrate, such that the photodiodes result in an array of pixels, as is indicated in FIG. 3 by the array division 204. However, the semiconductor substrate within which the photodiode array 112 is formed, is facing radiation 202 and/or distal end 69 with a main side which is opposite that main side of this semiconductor substrate within which the photodiode array is actually formed within this semiconductor substrate. Photons 202, which impinge on object 44 after backscattering, thus initially enter into the semiconductor substrate through the main side 204 of the semiconductor substrate of the photodiode array 112 so as to impinge, after passing through, on the photodiode array in that main side of the semiconductor substrate which is opposite the main side 204, or to impinge onto the space-charge regions and there to be converted to pixel measurement signals there by means of diffusion and/or drift current.

Using flip-chip bonding as an example of a method of structural design and coupling technology, the photodiode array 112 thus formed is disposed onto a semiconductor chip, such as a CMOS chip 114, which has the pre-processing means 50 integrated therein. Photodiode array 112 and chip 114 are connected to each other such that the main side of the semiconductor substrate within which the photodiode array 112 is formed faces the semiconductor chip 114 with that main side within which the photodiode array 112 is formed, i.e. with the side facing away from the main side 204, or with that main side which is facing away from the distal end. Semiconductor chip 114, in turn, is connected to photodiode array 112 such that it faces same with that main side of the chip within which the circuit which forms the drive, readout, and pre-processing electronics 50 is integrated. FIG. 3 also depicts cables 80 of FIG. 1 which are responsible for supplying compound arrangement 200 with energy and/or for passing on the processed data from chip 114 to image processing means 62 or, conversely, for passing on control signals from processing means 62 to chip 114, or to the circuit integrated thereon.

A specific configuration of a video endoscope in accordance with all of the previous embodiments of FIGS. 1 to 3, i.e. of a video endoscope exhibiting the structure of FIG. 1, the catheter tip of FIG. 2, and the photodetector array/pre-processing chip compound arrangement of FIG. 3, including an adaptation for cardiovascular examination, could comprise the following: as the external radiation source, an infrared diode; as a radiation router 70, several monomode fibers which adduct the radiation to the examination location 44; as feed lines 80 and 82, cables for supplying the pixel array/pre-processing compound arrangement with energy, and for reading out data; as an image sensor 48, a detector array 112 on a III-V semiconductor which is deposited, e.g. by means of flip-chip bonding, onto readout, pre-processing, and drive electronics integrated on an underlying CMOS chip 114; as optics 46, a lens system for optical imaging with the necessary depth of focus and a sufficient field of view with, as the case may be, autofocus; as the processing means, a processor 62 for image processing; and as monitor 64, a TFT monitor, for example, of which the latter two are, e.g., built into an external module 30 and drive the image sensor 48 via cables 80; and as possible further auxiliary apparatus such for controlling and/or turning the distal catheter end, i.e. a navigation aid, as well as possibly several sensors 52 for the purpose of further diagnostics and control, such as for the blood flow, the temperature, etc. A catheter which is miniaturized in such a manner and which adducts the image sensor 48, the optics 46, the monomode fibers for illumination, and the cables to the location of examination through the artery and/or vein systems should be biocompatible and encapsulated in a stable manner. This applies, in particular, to sheath 102, i.e. it should be biocompatible and sterile.

A cardiovascular video endoscope formed in such a manner considerably simplifies planning, implementation and subsequent monitoring of medical interventions within the vascular system of humans. Defects of the cardiovascular system may herewith be evaluated directly within a blood-filled environment. Due to the reduced intervention time, this results in a treatment which is overall more gentle on patients. Once the method has become well-established, the cost for treatment may be drastically reduced. In comparison with prior diagnostic systems, an endoscope formed in such a manner provides a clearly higher image resolution. Using the methods of modern image processing, such as pattern recognition which is performed, for example, within processing means 62 or within a different processor unit which has access to memory 66, any information desired on the part of the physician may be immediately derived from the data obtained by means of the catheter.

As has already been described above, sensor elements 52 are not absolutely necessary. Examples of such sensory elements which extend the distal end of the endoscope within the catheter tip in accordance with the user's requirements include a flow sensor, a temperature sensor, chemical sensors or the like.

Compared to the method of U.S. Pat. No. 6,178,346 which was mentioned in the introduction to the description, a video endoscope in accordance with the present invention comprises in-situ mounting of the camera device and/or the image sensor. From that point of view, image transmission by means of optical-fiber cables may also be dispensed with. Since such cables exhibit a lower aperture and, in addition, attenuate the optical signal, the image quality is comparatively poor with the conventional method. The above-described embodiments, by contrast, promise to achieve a considerably improved image quality.

An endoscopy device in accordance with the previous embodiments which is to be suitable for cardiovascular examination should operate at a wavelength of 2.1 μm, unlike conventional video endoscopes which exploit the visible wavelength range of 400-700 nm. Both our own theoretical calculations and experimental investigation confirm that blood is sufficiently transparent at this wavelength. The choice of wavelength is the result of a compromise: at low wavelengths, scattering of light at the particles is too high, at higher wavelengths, the absorption is too high due to the high proportion of water. The visibility range that can be achieved amounts to about 12 mm in blood at this wavelength. What is also feasible is a video endoscope which operates at a wavelength of 1.7 μm. In this case, the achievable visibility range would amount to 8 mm. Other wavelength ranges, such as from 1.5 μm to 1.8 μm, or from 2.1 μm to 2.3 μm, may also be sufficient, however.

Put differently again, the overall architecture, proposed above with reference to FIG. 1, of the video endoscopy device comprises a radiation source, a cable with feed lines and monomode fibers to enable illumination at the site in question, an image sensor comprising electronics, optics, a processor, a monitor and possibly further control devices and sensors. In accordance with a specific configuration, the video endoscope could comprise a miniaturized, encapsulated catheter head as is shown, for example, in FIG. 2. In addition to the image sensor array shown in FIG. 2, the optics, the readout and drive electronics, the interfaces and the illumination unit, it could also comprise further image sensor arrays. For example, using the additional image sensors, the image field may be enlarged, on the one hand, by laterally integrating the additional image sensors, for example, into the distal catheter tip, and on the other hand, the additional image sensors could employ different imaging methods, so that further and/or additional information is obtained via the respective different imaging methods. These imaging methods include, for example, laser-induced fluorescence (LIF) or the scattered-light method. For detailed examination, the area to be examined could be stained, for example, or be enriched with a specific substance, so that the reflective behavior locally changes when illuminated. In this manner, it would be easier to differentiate between different surface structures.

In addition, it is possible to directly integrate light sources, such as photodiodes, into the distal end of the catheter head, rather than using an externally arranged light source. These could then, in the embodiment of FIG. 2, be arranged, for example, at those locations which correspond to the exit points of the light guides 104 there. Instead of light guides 104, one would only need electrical feed lines for supplying the photodiodes and/or light sources with the power required.

As has been described with reference to FIG. 3, in accordance with one configuration, the image sensor array could be formed on the basis of a III-V semiconductor, for example as an InGaAs photodiode array, and be deposited onto a CMOS chip using flip-chip bonding. The underlying CMOS chip could include the readout, pre-processing, and drive electronics for the upper chip. The underlying CMOS chip could also have the interface electronics integrated therein, with which the acquired image signals can be transmitted, via the cable, to the processor within the external apparatus. Actual signal and image processing is executed within the external processor with a user-friendly interface, before the images are displayed on a monitor.

Since the external diameter is limited by the vessels—the larger arteries and/or veins have diameters of between 6 and 14 mm—the structure of the catheter of the above embodiments should be sufficiently miniaturized in the implementation. The minimum photodiode pitch is predefined by the diffraction-limited resolution, at 7 μm, so that a video endoscope having a diameter of 1.5 mm theoretically could offer a resolution of 20,000 picture elements (pixels).

The optics, or the optical system, should enable a visual range of at least 25 degrees. The optical system should autofocus within an image-width range from 5 to 12 mm. The lens diameter should not exceed 3 mm. The image rate of the image sensor should be at least 15 images per second.

A catheter head in accordance with the embodiments of FIGS. 2 and 3 could be employed by a physician in such a manner that the catheter is placed, by the physician, at the location to be examined. Once the examination location has been homogeneously illuminated with infrared radiation, which is passed from the infrared diode through the catheter into the body via monomode fibers, the image of the vascular walls is reproduced onto the image sensor by means of the optics, or the optical system. The radiation enters into the photodiode array through the substrate 112. This back-illumination has the advantage, on the one hand, as has already been mentioned with regard to FIG. 3, that the optical interface does without metal contacts, and has the advantage, on the other hand, that further optics may be monolithically integrated, for beam focusing, into the substrate of pixel array 112, i.e. in that part of array 112 which faces the distal end of the catheter and which is located between the distal end and the surface of the pixel array substrate, within which surface the pixel diodes of the pixel array are formed, so that the photosensitivity of each pixel could be increased in that the integrated optics focus radiation onto the photosensitive zones of the pixel diodes, i.e. the space-charge regions. Once the photons have been converted to charge/signals, these are read out, pre-processed and/or encoded via the CMOS chip, and are transmitted, via cables, to the external processor for image processing. The image processing unit extracts the information desired before it is presented on the monitor. Subsequently, this information is stored within a patients database, such as in memory 66.

It shall be noted that even though, in accordance with the above-described embodiments, a pre-processing means 50 has always already been arranged within the catheter head, it would also be possible to perform this pre-processing only within the framework of image processing within image processing means 62. Performing the pre-processing already within the catheter head, such as dynamics adjustment, channel adjustment, filtering out or source encoding, however, may possibly reduce the demands made on the routing of the pixel information and/or pixel measurement values to the external apparatus 30, such as reduce the number of cables required, or the like, or increase the transmission rate with the cabling unchanged.

As has already been mentioned above, arranging further sensors is not essential to the present invention. Conversely, as has also already been mentioned above, further devices, such as ones for navigating the endoscope within the blood vessels, may be provided within the catheter. To this end, one or more actuators which may—as the situation may be—be of mechanical nature, may be provided, which is why a mechanical Bowden control, which extends from the proximal end to the catheter so as to be able to control this actuator, may also be provided within the catheter.

In addition, it shall be pointed out that it is by way of example only that the previous embodiments referred to the representation of the cardiovascular system, i.e. to a cardiovascular endoscope for representing the interior walls of the cardiovascular system by means of a minimally invasive imaging system. Inventive video endoscopes, however, may also be employed in other places in medical diagnosis.

The preceding embodiments could be employed as an angioscope and could support, as a diagnostic tool, the heart and vascular surgeon in heart surgery to be performed with minimum invasiveness, such as in reconstructing and/or replacing mitral or tricuspidal valves, in obstructing an interventricular septal defect or in implanting coronary bypasses. In addition, various defects of the vascular system, e.g. lesions, aneurisms, scleroses and stenoses, can be made visible and evaluated pre-operatively. As far as intra-operative employment is concerned, the removal of these effects, e.g. by implanting a stent or by HF, or high frequency, ablation or cryoablation, may be accompanied with an angioscope. These interventions can be very readily evaluated post-operatively. A further large area of application of the embodiments described above is the exact evaluation of thromboses, embolisms and infarcts, which nowadays represents a challenge in a society with increasingly older patients. The improvement in the examination increases the safety in ensuing therapy.

Thus, above embodiments form a diagnostic tool and enable a diagnostic method associated therewith by means of which observations at and/or in organs and vessels may be performed pre-, intra-, and post-operatively in an actual, i.e. blood-filled, environment. The physician is able to look into the cardiovascular system through the catheter, the distal end of which he/she adducts to the examination location via the blood vessels, and through the extra-corporal image processing unit within the monitor. These video endoscopy devices support the surgeon performing treatment in navigating and performing difficult operations, for example on the heart. The above configurations enable novel diagnostic methods which, in turn, allow simple morphological-functional imaging of the cardiovascular system with variable application possibilities, and which accompany the physician both pre-, intra-, as well as post-operatively. Unlike the standard imaging methods used for representing the cardiovascular system, this imaging method provides a higher resolution without ionizing radiation.

While this invention has been described in terms of several preferred embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

What is claimed is:

1. A video endoscopy device comprising:
a sensor device; and
a catheter for outputting radiation at a distal end of the catheter, and for receiving reflected radiation at the distal end, and imaging same onto the sensor device,
the sensor device being arranged, within the catheter, in the vicinity of the distal end of the catheter, and is configured to convert the radiation reflected into an electric signal, and the catheter being configured to route the electric signal to a proximal end of the catheter.

2. The device as claimed in claim 1, wherein the catheter is further configured to route the radiation from the proximal end to the distal end of the catheter.

3. The video endoscopy device as claimed in claim 1, wherein the sensor device comprises a photodetector array.

4. The video endoscopy device as claimed in claim 1, wherein the catheter comprises a radiation router for routing the radiation from the proximal end to the distal end.

5. The video endoscopy device as claimed in claim 1, wherein the catheter further comprises optics for flaring the radiation.

6. The video endoscopy device as claimed in claim 1, wherein the sensor device is arranged in a manner which is essentially axially central to a longitudinal axis of the catheter, and wherein the radiation router extends from the proximal end of the catheter along the outer jacket of the catheter, past the sensor device, and to the distal end of the catheter.

7. The video endoscopy device as claimed in claim 6, wherein the radiation router is formed by a plurality of monomode fibers arranged, in cross section transverse to the longitudinal axis of the catheter, in an annular manner along the outer jacket of the catheter.

8. The video endoscopy device as claimed in claim 1, wherein the catheter comprises a light source integrated at the distal end of the catheter to output the radiation at the distal end of the catheter.

9. The video endoscopy device as claimed in claim 1, wherein the catheter comprises imaging optics for imaging the reflected radiation onto the sensor device.

10. The video endoscopy device as claimed in claim 7, wherein the catheter comprises imaging optics for imaging the reflected radiation onto the sensor device, and
wherein the imaging optics are arranged, within the catheter, in a manner which is essentially axially central to a longitudinal axis of the catheter, at the distal end of the catheter and are mounted, by means of fixtures, to the outer jacket of the catheter, the monomode fibers extending through the fixtures.

11. The video endoscopy device as claimed in claim 8, wherein the radiation source comprises an infrared diode.

12. The video endoscopy device as claimed in claim 1, wherein the catheter comprises an energy supply for supplying the sensor device with energy.

13. The video endoscopy device as claimed in claim 1, further comprising an image processor for processing the electric signal to obtain an endoscopy image, the image processor being adapted to be coupled to the proximal end of the catheter.

14. The video endoscopy device as claimed in claim 1, wherein the sensor device comprises pre-processing electronics for pre-processing the electric signal.

15. The video endoscopy device as claimed in claim 14, wherein the pre-processing electronics are adapted such that they include dynamics adjustment, channel adjustment, noise filtering or source encoding.

16. The video endoscopy device as claimed in claim 1, further comprising a monitor adapted to be coupled to the image processor.

17. The video endoscopy device as claimed in claim 1, further comprising a sensor element, arranged within the catheter, for detecting pressure, temperature or a pH value.

18. The video endoscopy device as claimed in claim 1, wherein the sensor device is operative at an operation wavelength of between 1.5 μm and 1.8 μm, or between 2.1 μm and 2.3 μm.

19. The video endoscopy device as claimed in claim 1, wherein the sensor device comprises a photodiode array arranged on a main side of a semiconductor substrate, the photodiode array being arranged, within the catheter, such that the main side of the photodiode array faces away from the distal end, and that a main side of the semiconductor substrate which is opposite said main side faces the distal end.

20. The video endoscopy device as claimed in claim 19, wherein the sensor device comprises:
a chip for signal processing, the chip being connected to the photodiode array by means of flip-chip bonding, such that the main side of the semiconductor substrate, wherein the photodiode array is formed, faces the chip.

21. The video endoscopy device as claimed in claim 20, wherein a signal processing circuit for pre-processing the electric signal is integrated within a main side of the chip, said main side facing the photodiode array.

22. The video endoscopy device as claimed in claim 19, wherein beam-focusing optics are integrated into the semiconductor substrate of the photodiode array.

23. The video endoscopy device as claimed in claim 1, suitable for cardiovascular endoscopy.

24. The video endoscopy device as claimed in claim 1, wherein an additional image sensor is provided, such that the image field is enlarged by the additional image sensor and sensor device together, compared to an image field of the sensor device alone, or that the additional image sensor is based on a different imaging method than the sensor device.
